(19) United States
(12) Patent Application Publication
(10) Pub. No.: US 2013/0118255 A1
(43) Pub. Date: May 16, 2013

(54) METHODS AND APPARATUS FOR CONFORMAL SENSING OF FORCE AND/OR CHANGE IN MOTION

(71) Applicants: Gilman Callsen, Malden, MA (US); Roozbeh Ghaffari, Cambridge, MA (US); William J. Arora, Boston, MA (US); Bassel de Graff, Don Miguel (TT); Benjamin Schlatka, Lexington, MA (US); Jeffrey D. Carbeck, Belmont, MA (US)

(72) Inventors: Gilman Callsen, Malden, MA (US); Roozbeh Ghaffari, Cambridge, MA (US); William J. Arora, Boston, MA (US); Bassel de Graff, Don Miguel (TT); Benjamin Schlatka, Lexington, MA (US); Jeffrey D. Carbeck, Belmont, MA (US)

(21) Appl. No.: 13/516,486

(22) PCT Filed: Dec. 17, 2010
(86) PCT No.: PCT/US10/61151

§ 371 Date: Jan. 22, 2013

Related U.S. Application Data
(60) Provisional application No. 61/287,615, filed on Dec. 17, 2009.

(30) Foreign Application Priority Data
Oct. 1, 2010 (US) PCT/US2010/051196

(51) Int. Cl. A61B 5/11

(52) U.S. Cl. CPC A61B 5/11
USPC 73/491

(57) ABSTRACT

Sensing a force and/or a change in motion proximate to an arbitrarily-shaped surface via a conformal sensing element (e.g., a pressure sensor, an accelerometer) disposed on a flexible substrate and having a sufficient mechanical coupling to the surface. The conformality of the sensing element facilitates intimate proximity to the surface to ensure accurate sensing. Examples of arbitrarily-shaped surfaces include body parts of a person (e.g., a head). A processor receiving one or more signals from the sensing element may provide information relating to possible injury to a body part (e.g., head trauma) resulting from sensed forces and/or changes in motion. Such information may be conveyed by one or more output devices that provide indications of possible degrees of injury/trauma. A conformal sensing apparatus may be integrated with a protective garment or accessory, such as a helmet, wherein the conformality of the sensing apparatus also ensures sufficient comfort for the wearer.

### PRIORITY APPLICATIONS

The present application claims a priority benefit to U.S. provisional patent application Ser. No. 61/287,615, filed Dec. 17, 2009, entitled “Conformal, Helmet-Pad Integrated Blast Dosimeter.”

The present application also claims a priority benefit to PCT application no. PCT/US2010/051196, filed Oct. 1, 2010, entitled “Protective Cases with Integrated Electronics.”

Each of the above-identified applications is hereby incorporated herein by reference in its entirety.

### BACKGROUND

A motion (e.g., a translation and/or a rotation), or a sudden change in motion (e.g., an acceleration and/or change in orientation) of a person's body part potentially can cause injury to the person. Sudden changes in motion typically result from an appreciable force exerted (e.g., an impact) proximate to or on some portion of the person's body, and may arise in a variety of contexts, such as in connection with sporting or other recreational activities, work-related activities, vehicle-related activities and incidents (e.g., collisions, crashes, reckless operation), and military activities (e.g., training, combat).

For example, in sporting activities such as football, soccer, boxing, hockey, baseball, basketball and the like, participants may bump into each other, push or punch each other, fall to the ground, hit an obstacle or other object, and/or be hit by a ball, puck, bat, or other object. Other activities such as running or jumping may result in slips or falls involving impact on, and/or unconventional movement of, one or more body parts. Similarly, in any of a wide variety of workplace environments, a person may slip, fall and impact the ground or another object, or be struck by an object in the environment; for example, in a workplace environment such as a construction site, a worker may be hit by falling debris/construction materials. In other working environments involving potentially volatile materials (e.g., chemical plants, refineries, mining activities), workers and others present may be subject to an unexpected explosion (a “blast”). Likewise, in military training or combat situations, a soldier may find themselves in the vicinity of an explosion and be subject to the force associated with same. Any of the foregoing illustrative situations may result in some degree of injury (or “trauma”) to the person.

In the illustrative contexts mentioned above, a variety of accessories or garments may be worn by a person to provide protection of various body parts against injury. Particular attention often is paid to protecting a person's head in connection with potentially injurious activities. To this end, a helmet is a well-known accessory that may be a worn by a person to protect against head trauma. Many types of helmets exist for various applications, including sporting and recreational use, work use, vehicle use, and military use. Other examples of accessories or garments worn by a person to provide various degrees of protection include footwear, gloves, vests, jackets, and other types of clothing.

Examples of specialized accessories or garments, including helmets, are known that include various sensing devices. An “accelerometer” is an exemplary sensing device that is configured to sense a change in motion. Generally speaking, a change in motion may refer to one or more of an acceleration (i.e., a change in velocity), a change in orientation, a vibration shock, and a falling process. Conventional accelerometers are capable of sensing various changes in motion along one or more axes. An accelerometer typically provides an output signal representative of a “g-force” acting on an object that is free to move (i.e., the “g-force” is the object's acceleration relative to free-fall due to the vector sum of non-gravitational forces acting on the object). A g-force (denoted by the unit g) causes stresses and strains on an object, and hence large g-forces may be destructive. Some types of commercially available (i.e., “commercial off-the-shelf,” or “COTS”) accelerometers may include piezoelectric or capacitive components to convert mechanical motion into an electrical signal. Piezoelectric accelerometers rely on piezoceramic materials or single crystals, whereas capacitive accelerometers typically employ a silicon micro-machined sensing element (a micro-electrical-mechanical system, or MEMS, sensing element).

Some examples of protective garments including sensors are described in U.S. Pat. No. 7,150,048. The garments discussed in this patent primarily are related to providing padding and impact cushioning for the wearer in the form of inflatable garments, in which the inflatable garments are activated to inflate based on sensed ballistic parameters such as acceleration, distance, relative acceleration, and rotation. Disclosed examples of such protective garments include shorts, pants, jackets, vests, and underwear. It is noteworthy that the use of sensors in this patent relates specifically to activation of inflatable garments, and is not related to sensing changes in motion of one or more body parts in connection with possible injury to same.

Other examples of garments or personal accessories including sensors are described in U.S. Pat. Nos. 7,054,784 and 7,457,724. The sensing units described in these patents primarily relate to sporting activities in which the sensing unit may be integrated with a ski or snowboard, boots and bindings for same, roller blades, a skate-board, a mountain bike, a windsurfer, a kayak, etc. The sensing units collect and store “performance data” including airtime, speed, and drop distance. The sensing units may wirelessly communicate with a watch worn by a person so as to display performance data or other sporting characteristics. In one example, a sensing unit for a shoe includes an accelerometer and a processor to determine one or both of speed and distance traveled of a person wearing the shoe. As with the inflatable garment patent discussed above, the use of sensors in this patent is not related to sensing changes in motion of one or more body parts in connection with possible injury to same, but rather to collect various performance data relating to a sporting activity so as to assess the activity.

With respect particularly to protective helmets, U.S. Pat. No. 4,763,275 is directed to a sports helmet equipped with a sensor mounted on an outer surface of the helmet to sense forces or vibrations and determine the accumulative force encountered by the helmet over a period of time. U.S. Pat. No. 5,539,935 is directed to a sports helmet that includes an integrated sensor and signaling device mounted on an outer surface of the helmet (e.g., to the rear of either a left or right ear hole) for detecting impacts above a selected magnitude and for providing a signal representing the magnitude and direction of an impact. U.S. Pat. No. 5,978,972 is directed to a helmet system (e.g., a boxing helmet) including three or more accelerometers embedded inside the material of the helmet to measure and record in real time data relating to acceleration of an individual's head during normal sporting activity. U.S. Pat. No. 7,509,835 is directed to a helmet having a shock detector that includes an accelerometer employing liquid surface tension technology, which is mounted to an outer surface of the helmet. The shock detector provides a visual signal when the helmet is subjected to an impact exceeding a predetermined threshold level. U.S. Pat. Nos. 6,826,509 and 7,526,389 are directed to a helmet that is equipped with multiple accelerometers oriented with respect to each other to sense linear acceleration orthogonal to the outer surface of the skull. In one disclosed implementation, the accelerometers are contained in an air bladder mounted within the helmet. In another disclosed implementation, the accelerometers are mechanically coupled to a T-shaped holder that is pressed against the skull.

### SUMMARY

Regarding sensing significant changes in motion of a person's head (e.g., resulting from a force exerted on or in proximity to the person's head), the Inventors have identified various shortcomings in connection with conventional apparatus for sensing such changes.

For example, regarding the use of multiple accelerometers or other sensors coupled to a helmet as described in several U.S. patents mentioned above, or a T-shaped holder as described in U.S. Pat. No. 6,826,509, the Inventors have appreciated that accelerometers or other sensors mounted on an outer surface of a helmet, within the material of a helmet, or contained in an air bladder mounted within the helmet, may not accurately sense significant changes in motion of the person's head. In particular, although such configurations may maintain a reasonable level of comfort for the person wearing the helmet (due to the sensors being mounted outside or within the material of a helmet, or the cushioning effect of an air bladder), the helmet or air bladder itself diminishes the degree of mechanical coupling between the accelerometers and the head. Accordingly, the accuracy of the accelerometers in sensing actual changes in head movement is reduced, and may include misleading components due to vibration or motion of the helmet itself. In contrast, pressing multiple accelerometers against the skull via a T-bar implementation may increase the mechanical coupling between the accelerometers and the skull and thereby increase the accuracy of sensing; however, this increased accuracy comes at the expense of significant discomfort to the person as well as possible safety hazards.

In view of the foregoing, the Inventors have recognized and appreciated that both sufficient comfort and accuracy are desirable attributes of techniques for sensing changes in motion of a person's head or other body part.

Beyond sensing changes of motion, the Inventors further have appreciated that alternatively or additionally sensing a force exerted on a person's head or other body part also may provide valuable information in connection with possible injury resulting from the force. In particular, by sensing both the onset of noteworthy changes in force acting on a person, and resulting changes in motion due to the force, an informative profile of a person's exposure and possible trauma resulting therefrom may be obtained.

Irrespective of the type and number of sensing elements employed to detect forces and/or changes in motion proximate to a person's head or other body part, the Inventors further have appreciated that a sensing element which substantially conforms to arbitrarily-shaped surfaces, such as those typically associated with body parts, would significantly facilitate both comfort and accuracy in sensing important parameters relating to possible injury. In particular, conformal sensing element form factors that facilitate a sufficient mechanical coupling to a surface of interest (e.g., associated with a body part such as the head), and thereby reduce vibration or motion components not specifically associated with the surface, significantly improve the accuracy of force and/or change in motion measurements.

Accordingly, various inventive embodiments disclosed herein relate to methods and apparatus for conformal sensing of force and/or change in motion.

In illustrative embodiments, sufficiently accurate sensing of force and/or change in motion at an arbitrarily-shaped surface, such as a surface typically associated with a body part of a person, is accomplished by an apparatus including a sensing element disposed on or otherwise integrated with a flexible substrate that substantially conforms to the arbitrarily-shaped surface. In one aspect, the conformable nature of the apparatus facilitates intimate proximity of the apparatus to the surface at which accurate sensing of force and/or change in motion is desired. “Intimate proximity” generally refers to a sufficient mechanical coupling to the arbitrarily-shaped surface without undesirable obstruction (e.g., the apparatus maintains a relatively low profile with respect to the surface), undesirable interference (e.g., from other motion or vibration not related to the surface), and/or compromise to comfort (e.g., in the case of a body part of a person). In some exemplary implementations discussed herein, intimate proximity is realized as substantial direct contact with the arbitrarily-shaped surface, due to the ability of the apparatus to conform to various contours of the surface.

Examples of significantly contoured and arbitrarily-shaped surfaces contemplated in connection with the inventive concepts disclosed herein, particularly in the context of body parts, include, but are not limited to: complex curvature due to muscle, tendon, bone (e.g., skull) and/or cartilage; sharply angled features at joints; compliance of skin; and perturbations on otherwise relatively flat sections of surface (e.g., veins on skin surface). It should be appreciated, however, that notwithstanding the foregoing examples relating primarily to body parts, a wide variety of arbitrarily-shaped surfaces, whether or not associated with a living creature, are contemplated in connection with the inventive concepts disclosed herein.

In some embodiments, a sensing apparatus may include one or more sensing elements (e.g., one or both of a pressure sensor and an accelerometer) disposed on or integrated with the flexible substrate. Additionally, in some embodiments the apparatus further may include one or more of a processor, a memory, a communication interface and a power source. In one aspect, one or more of the processor, the memory, the communication interface and the power source also may be disposed on or integrated with the flexible substrate. In another aspect, the processor may receive and/or process one or more output signals generated by the sensing element(s) and, in the context of sensing force and/or change in motion proximate to a body part, provide information relating to possible injury/trauma based at least in part on the output signal(s). In yet other aspects, the memory may store various data relating to the output signal(s) provided by the sensing element(s), and the communication interface may communicate various information to and/or from the apparatus.

Some inventive embodiments discussed in further detail below relate to a system of a conformal sensing apparatus and one or more output devices to provide perceivable indicators or cues (e.g., audible cues, visual cues) representing impact or trauma based on sensed forces and/or changes in motion. For example, in one embodiment, one or more acoustic speakers, and/or one or more light sources, are coupled to a conformal sensing apparatus to provide one or more audible and/or visual cues representing impact or trauma, based at least in part on one or more output signals generated by the sensing apparatus. In one exemplary implementation involving visual cues, multiple light emitting diodes (LEDs) having different colors are employed, wherein different colors of LEDs, when energized, respectively correspond to different degrees of the impact or potential trauma.

To facilitate conformality of a sensing apparatus according to various embodiments disclosed herein, the flexible substrate of a conformal sensing apparatus may be formed of a plastic material or an elastomeric material, including any of a wide variety of polymeric materials. In one embodiment, the flexible substrate is configured as a flexible “tape” (e.g., having a thickness of less than five millimeters) that may have an adhesive disposed on at least one surface of the tape (to render the tape “sticky”). The form factor of an adhesive tape in some implementations facilitates integration of the sensing apparatus with any of a wide variety of protective garments and accessories while at the same time ensuring appreciable comfort and sensing accuracy, as discussed in greater detail below.

In some embodiments, one or more of the various functional components of a sensing apparatus according to the inventive concepts disclosed herein may be a commercial off-the-shelf (COTS) component (e.g., a pre-packaged chip) that is disposed on or integrated with the flexible substrate. In other embodiments, one or more functional components may be particularly-fabricated, and disposed on or integrated with the flexible substrate at a die level.

In yet another embodiment, to facilitate the conformal nature of the sensing apparatus, some or all of the functional components disposed on or integrated with the flexible substrate may be electrically coupled to each other using one or more flexible and/or stretchable interconnects. Flexible and/or stretchable interconnects may employ metals (e.g., copper, silver, gold, aluminum, alloys) or semiconductors (e.g., silicon, indium tin oxide, gallium arsenide) that are configured so as to be capable of undergoing a variety of flexions and strains (e.g., stretching, bending, tension, compression, flexing, twisting, torqueing), in one or more directions, without adversely impacting electrical connection to, or electrical conduction from, one or more functional components of the sensing apparatus. Examples of such flexible and/or stretchable interconnects include, but are not limited to, wavy interconnects, bent interconnects, buckled interconnects, and serpentine patterns of conductors.

In other embodiments, a sensing apparatus for conformal sensing of force and/or change in motion may be integrated into a protective garment or accessory to be worn by a person, such as a helmet. In particular, in one embodiment, a helmet includes one or more suspension pads to facilitate a safe, comfortable and tight fit of the helmet to a person's head. The helmet also may include one or more pad coupling mechanisms (e.g., Velcro® brand hook-and-loop fasteners) to detachably couple the suspension pad(s) to the helmet (e.g., so as to facilitate flexible placement of the suspension pad(s) on the helmet). In one aspect of this embodiment, a sensing apparatus having one or more of the features described herein is integrated with a suspension pad such that when the helmet is worn by a person, the sensing apparatus is in comfortable and safe intimate proximity to the person's head (e.g., to accurately sense a force and/or a change in motion at a surface of the person's head). In various examples, such helmets having sensing apparatus integrated therewith may be configured for various applications (e.g., sporting and recreational uses, workplace activities, vehicular-related use, military use, etc.).

In one aspect of embodiments relating to protective garments or accessories such as helmets, a sensing apparatus for conformal sensing of force and/or acceleration further may include switching circuitry to detect the proximity of the apparatus to a person, so that the apparatus is placed into a particular operational mode (e.g., powered-up) when proximity to the person is detected. In exemplary implementations, such switching circuitry may include one or more capacitive sensors to detect changes in electric field (e.g., due to an electrical conductivity of a person's skin) so as to sense proximity of the apparatus to the person.

In sum, one embodiment of the present invention is directed to an apparatus for sensing force and/or acceleration proximate to a person's head. The apparatus comprises a flexible substrate to substantially conform to a surface of the person's head so as to facilitate comfortable and safe intimate proximity of the apparatus to the person's head. The apparatus also comprises at least one sensing element disposed on the flexible substrate, the at least one sensing element including at least one of a pressure sensor and an accelerometer and generating at least one output signal. The apparatus further comprises a processor, disposed on the flexible substrate and communicatively coupled to the at least one sensing element, to receive and process the at least one output signal, and a memory, disposed on the flexible substrate and communicatively coupled to the processor, to store data relating to the at least one output signal.

Another embodiment is directed to a method, comprising sensing force and/or acceleration proximate to a person's head via at least one sensing element disposed on a flexible substrate in sufficient contact with the person's head, the flexible substrate substantially conforming to a surface of the person's head so as to facilitate comfortable and safe intimate proximity of the at least one sensing element to the person's head, the at least one sensing element including at least one of a pressure sensor and an accelerometer.

Another embodiment is directed to a helmet comprising at least one suspension pad and an apparatus, integrated with the at least one suspension pad, to sense a force and an acceleration proximate to a head of a person wearing the helmet. The apparatus comprises a flexible substrate to substantially conform to a surface of the person's head so as to facilitate comfortable and safe intimate proximity of the apparatus to the person's head, a pressure sensor and an accelerometer disposed on the flexible substrate and generating at least one output signal, a processor communicatively coupled to the pressure sensor and the accelerometer to receive and process the at least one output signal so as to provide information relating to a blast exposure, and a power source. The apparatus further comprises switching circuitry, disposed on the flexible substrate and electrically coupled to the power source, to detect the proximity of the apparatus to the person's head and to couple and/or decouple the power source and at least the processor based at least in part on the detected proximity of the apparatus to the person's head.

Another embodiment is directed to an apparatus for sensing a change in motion proximate to an arbitrarily-shaped surface. The apparatus comprises a flexible substrate to substantially conform to the arbitrarily-shaped surface so as to facilitate intimate proximity of the apparatus to the arbitrarily-shaped surface, and a single sensing element disposed on the flexible substrate to sense the change in motion proximate to the arbitrarily-shaped surface.

Another embodiment is directed to a method, comprising sensing a change in motion proximate to an arbitrarily-shaped surface via a single sensing element disposed on a flexible substrate having a sufficient mechanical coupling to the arbitrarily-shaped surface.

Another embodiment is directed to a system comprising an apparatus for sensing a change in motion proximate to an arbitrarily-shaped surface. The apparatus comprises a flexible substrate to substantially conform to the arbitrarily-shaped surface so as to facilitate intimate proximity of the apparatus to the arbitrarily-shaped surface, and a sensing element disposed on the flexible substrate to sense the change in motion proximate to the arbitrarily-shaped surface, wherein the change in motion includes at least one of an acceleration, an orientation, a vibration shock and a falling process. The sensing element includes an accelerometer, a processor communicatively coupled to the accelerometer, and a memory communicatively coupled to the processor. The system further includes a coupling mechanism to mechanically couple the apparatus to the arbitrarily-shaped surface, and a plurality of LEDs coupled to the sensing element to provide at least one visual cue representing impact or trauma based at least in part on at least one output signal generated by the accelerometer, wherein the plurality of LEDs have different colors respectively corresponding to a degree of the impact or trauma.

Another embodiment is directed to an apparatus for sensing a change in motion proximate to a body part of a person. The apparatus comprises a flexible substrate to substantially conform to a surface of the body part so as to facilitate comfortable and safe intimate proximity of the apparatus to the body part, at least one sensing element disposed on the flexible substrate to sense the change in motion proximate to the body part and provide at least one output signal representing the sensed change in motion, and a power source to provide power to the at least one sensing element. The apparatus further comprises switching circuitry, coupled to the power source and the at least one sensing element, to detect the proximity of the apparatus to the body part and to electrically couple and/or decouple the power source and the at least one sensing element based at least in part on the detected proximity of the apparatus to the body part.

Another embodiment is directed to a method, comprising: A) sensing a change in motion proximate to a body part of a person via at least one sensing element disposed on a flexible substrate having a sufficient mechanical coupling to the body part; B) detecting a proximity of the at least one sensing element to the body part; and C) coupling and/or decoupling power to and/or from the at least one sensing element based at least in part on B).

Another embodiment is directed to a system, comprising an apparatus for sensing a change in motion proximate to a body part of a person. The apparatus comprises a flexible substrate to substantially conform to a surface of the body part so as to facilitate comfortable and safe intimate proximity of the apparatus to the body part. The apparatus further includes at least one sensing element disposed on the flexible substrate to sense the change in motion proximate to the body part and provide at least one output signal representing the sensed change in motion, a processor communicatively coupled to the at least one sensing element, a memory communicatively coupled to the processor, and a power source to provide power to at least the at least one sensing element. The apparatus further includes switching circuitry, coupled to the power source and the at least one sensing element, to detect the proximity of the apparatus to the body part and to electrically couple and/or decouple the power source and the at least one sensing element based at least in part on the detected proximity of the apparatus to the body part. The system further includes a coupling mechanism to mechanically couple the apparatus to the body part, and at least one light source coupled to the processor to provide at least one visual cue representing impact or trauma based at least in part on the at least one output signal generated by the at least one sensing element.

Another embodiment is directed to an apparatus for sensing a force and/or a change in motion proximate to a person's head and providing information relating to possible head trauma of the person. The apparatus comprises a flexible substrate to substantially conform to a surface of the person's head so as to facilitate comfortable and safe intimate proximity of the apparatus to the person's head, at least one sensing element disposed on the flexible substrate to sense the force and/or the change in motion proximate to the person's head and provide at least one output signal representing the sensed force and/or change in motion, and a processor communicatively coupled to the at least one sensing element to receive the at least one output signal and provide information relating to the possible head trauma of the person based at least in part on the at least one output signal.

Another embodiment is directed to a method, comprising: A) sensing a force and/or a change of motion proximate to a person's head via at least one sensing element disposed on a flexible substrate having a sufficient mechanical coupling to the person's head; and B) electronically providing information relating to possible head trauma of the person based at least in part on A).

Another embodiment is directed to a system, comprising an apparatus for sensing an acceleration proximate to a person's head and providing information relating to possible head trauma of the person. The apparatus comprises a flexible substrate to substantially conform to a surface of the person's head so as to facilitate comfortable and safe intimate proximity of the apparatus to the person's head, at least one accelerometer disposed on the flexible substrate to sense the acceleration proximate to the person's head and provide at least one output signal representing the sensed acceleration, and a processor communicatively coupled to the at least one accelerometer to receive the at least one output signal and provide information relating to the possible head trauma of the person based at least in part on an acceleration curve associated with the at least one output signal. The system further comprises at least one light source coupled to the processor to provide at least one visual cue representing the information relating to the possible head trauma, wherein the at least one light source includes a plurality of LEDs having different colors respectively corresponding to a degree of the possible head trauma.

The following publications are hereby incorporated herein by reference:

Kim et al., “Stretchable and Foldable Silicon Integrated Circuits,” Science Express, March 27, 2008, 10.1126/science. 1154367;

Ko et al., “A Hemispherical Electronic Eye Camera Based on Compressible Silicon Optoelectronics,” Nature, August 7, 2008, vol. 454, pp. 748-753;

Kim et al., “Complementary Metal Oxide Silicon Integrated Circuits Incorporating Monolithically Integrated Stretchable Wavy Interconnects,” Applied Physics Letters, July 31, 2008, vol. 93, 044102;

Kim et al., “Materials and Noncoplanar Mesh Designs for Integrated Circuits with Linear Elastic Responses to Extreme Mechanical Deformations,” PNAS, December 2, 2008, vol. 105, no. 48, pp. 18675-18680;

Meitl et al., “Transfer Printing by Kinetic Control of Adhesion to an Elastomeric Stamp,” Nature Materials, January, 2006, vol. 5, pp. 33-38;

U.S. publication No. 2010 0002402-A1, published Jan. 7, 2010, filed Mar. 5, 2009, and entitled “STRETCHABLE AND FOLDABLE ELECTRONIC DEVICES;”

U.S. publication No. 2010 0087782-A1, published Apr. 8, 2010, filed Oct. 7, 2009, and entitled “CATHETER BALLOON HAVING STRETCHABLE INTEGRATED CIRCUITRY AND SENSOR ARRAY;”

U.S. publication No. 2010 0116526-A1, published May 13, 2010, filed Nov. 12, 2009, and entitled “EXTREMELY STRETCHABLE ELECTRONICS;”

U.S. publication No. 2010 0178722-A1, published Jul. 15, 2010, filed Jan. 12, 2010, and entitled “METHODS AND APPLICATIONS OF NON-PLANAR IMAGING ARRAYS;” and

U.S. publication No. 2010 027119-A1, published Oct. 28, 2010, filed Nov. 24, 2009, and entitled “SYSTEMS, DEVICES, AND METHODS UTILIZING STRETCHABLE ELECTRONICS TO MEASURE TIRE OR ROAD SURFACE CONDITIONS.”

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

The foregoing and other aspects, embodiments, and features of the present teachings can be more fully understood from the following description in conjunction with the accompanying drawings.

### BRIEF DESCRIPTION OF THE DRAWINGS

The skilled artisan will understand that the figures, described herein, are for illustration purposes only. It is to be understood that in some instances various aspects of the invention may be shown exaggerated or enlarged to facilitate an understanding of the invention. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way.

FIG. 1 illustrates an apparatus for conformal sensing of a force and/or a change in motion proximate to a person's head, according to one embodiment of the present invention.

FIG. 2 illustrates a cross-section profile of the apparatus of FIG. 1 configured as a flexible tape, according to one embodiment of the present invention.

FIG. 3 illustrates a top view of the apparatus of FIG. 1 in which one or more functional components may be electrically connected by one or more flexible and/or stretchable interconnects, according to one embodiment of the present invention.

FIG. 4 illustrates a helmet including one or more suspension pads with which the apparatus of FIG. 1 may be integrated, according to one embodiment of the present invention.

FIG. 5 illustrates an exemplary suspension pad of the helmet of FIG. 4 having the sensing apparatus of FIG. 1 integrated therewith, according to one embodiment of the present invention.

FIG. 6 is a functional block diagram of the sensing apparatus of FIG. 1, according to one embodiment of the present invention.

FIGS. 7A and 7B illustrate a circuit diagram of the sensing apparatus of FIG. 1 corresponding to the block diagram of FIG. 6, according to one embodiment of the present invention.

FIG. 8 is a flowchart illustrating a method for conformal sensing of force and/or change in motion, according to one embodiment of the present invention.

### DETAILED DESCRIPTION

Following below are more detailed descriptions of various concepts related to, and embodiments of, inventive methods and apparatus for conformal sensing of force and/or change in motion. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

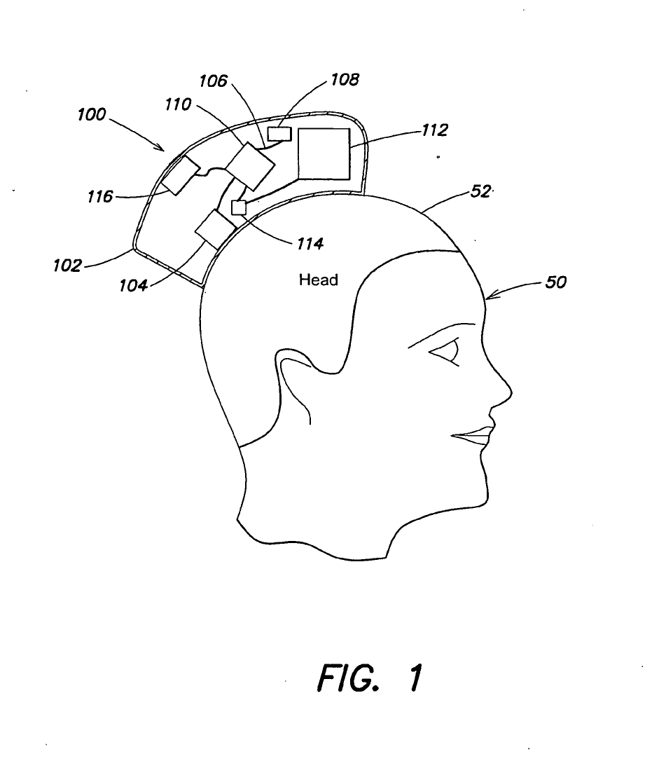

FIG. 1 illustrates an apparatus 100 for conformal sensing of a force and/or a change in motion proximate to a person's head 50, according to one embodiment of the present invention. The apparatus 100 comprises a flexible substrate 102 that facilitates a sufficient mechanical coupling of the apparatus 100 to a surface 52 of the person's head. In one aspect, the conformality of the apparatus provided at least in part by the flexible substrate 102 facilitates comfortable and safe intimate proximity to the surface 52 to ensure accurate sensing of force and/or change in motion in connection with the head 50. As noted above, “intimate proximity” generally refers to a sufficient mechanical coupling to a surface (e.g., the surface 52) without undesirable obstruction (e.g., the apparatus 100 maintains a relatively low profile with respect to the surface 52), undesirable interference (e.g., from other motion or vibration not related to the surface 52), and/or compromise to comfort. In some exemplary implementations discussed herein, and as shown for purposes of illustration in FIG. 1, intimate proximity may be realized as substantial direct contact with the surface 52, due to the ability of the apparatus to conform to various contours of the surface (e.g., based at least in part on the flexible substrate 102).

In the embodiment shown in FIG. 1, the flexible substrate 102 may include a plastic material or an elastomeric material. More generally, examples of materials suitable for purposes of the flexible substrate 102 include, but are not limited to, any of a variety of polyimides, polyesters, a silicone or siloxane (e.g., polydimethylsiloxane or PDMS), a photo-patternable silicone, an SU8 polymer, a PDS polydustrene, a parylene, a parylene-N, an ultrahigh molecular weight polyethylene, a polyether ketone, a polyurethane, a polyactic acid, a polyglycolic acid, a polytetrafluoroethylene, a polyamic acid, a polymethyl acrylate, and other polymers or polymer composites.

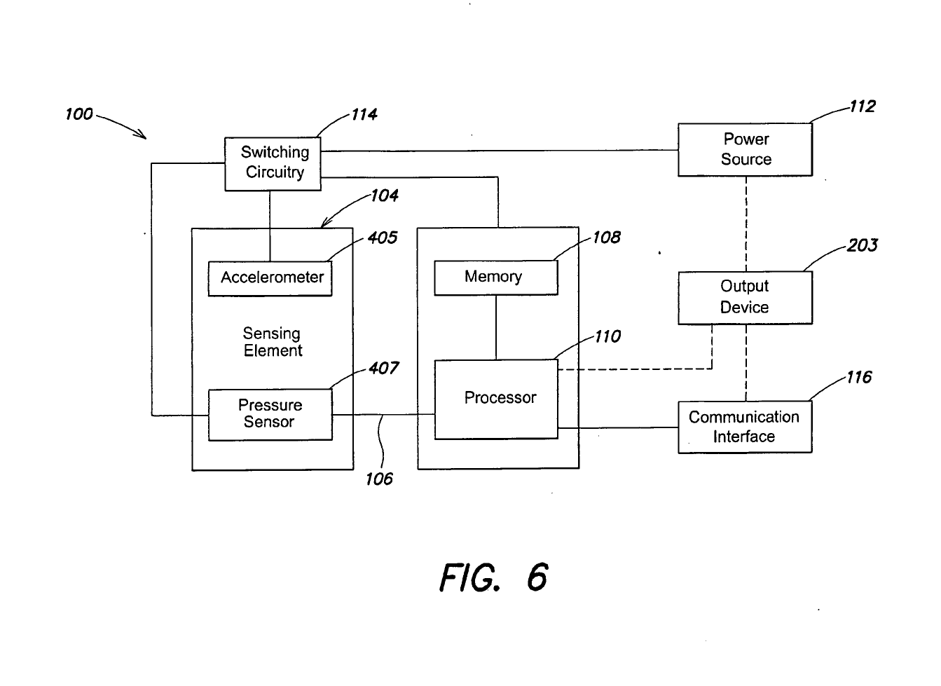

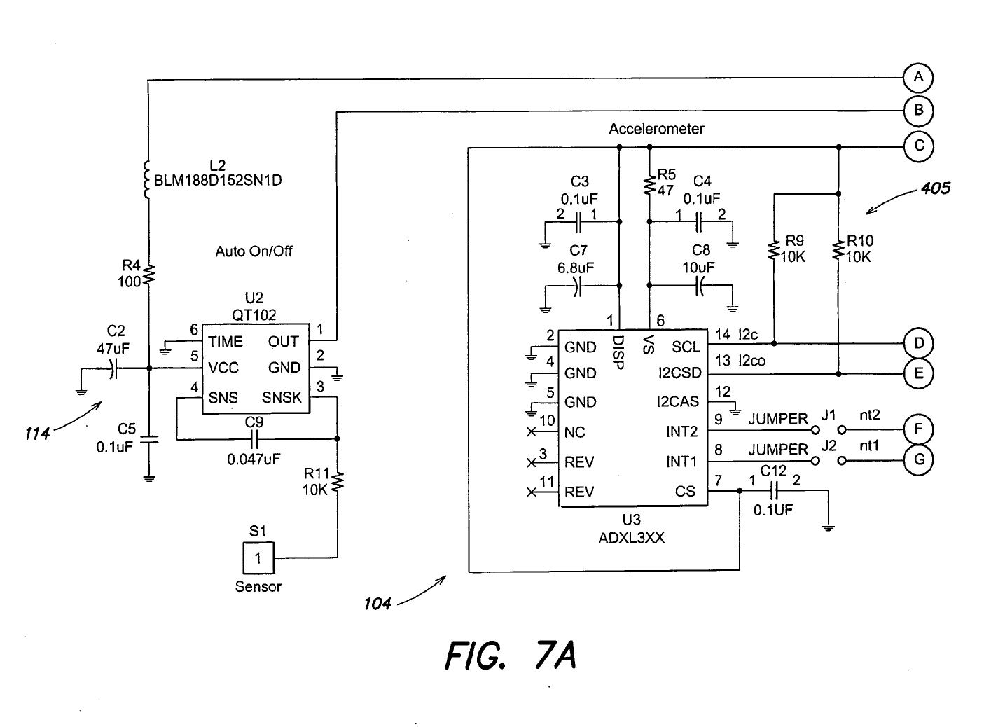

The apparatus 100 shown in the embodiment of FIG. 1 further includes one or more sensing elements 104, disposed on or otherwise integrated with the flexible substrate 102, to sense a force and/or a change in motion (e.g., an acceleration or change in velocity, a change in orientation, a vibration shock, a falling process). The sensing element(s) 104 generate one or more output signals 106 (e.g., representing sensed force and/or change in motion proximate to the head 50). As discussed further below in connection with FIG. 6, in exemplary implementations the sensing element(s) may include one or both of a pressure sensor and an accelerometer to accurately measure forces and/or changes in motion at or near the surface 52 of the head 50.

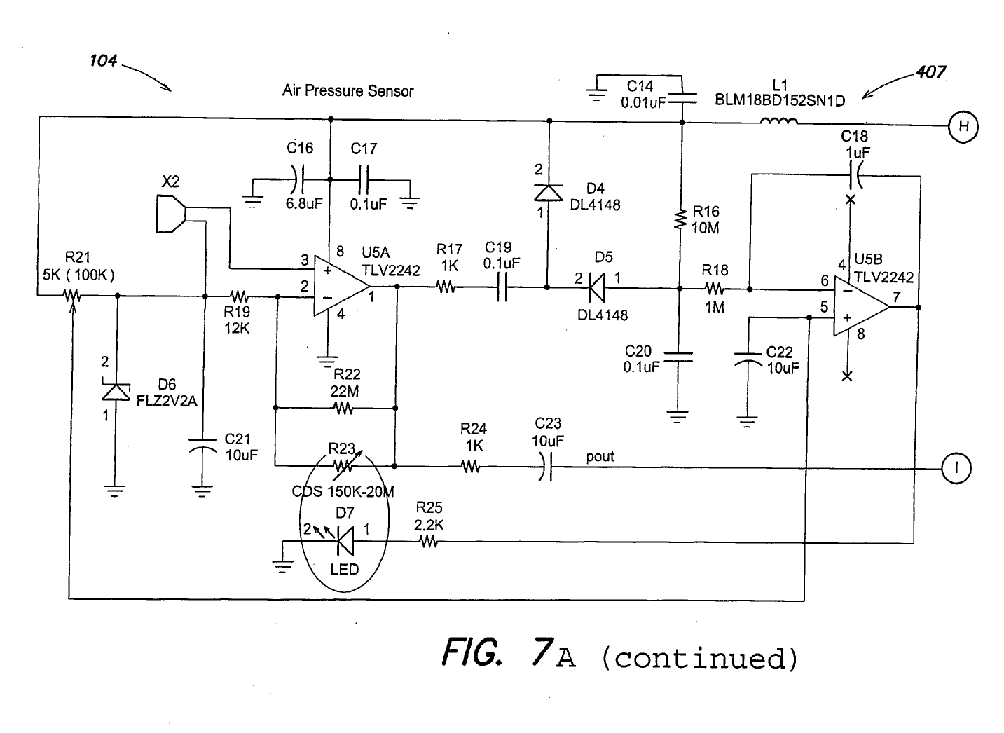

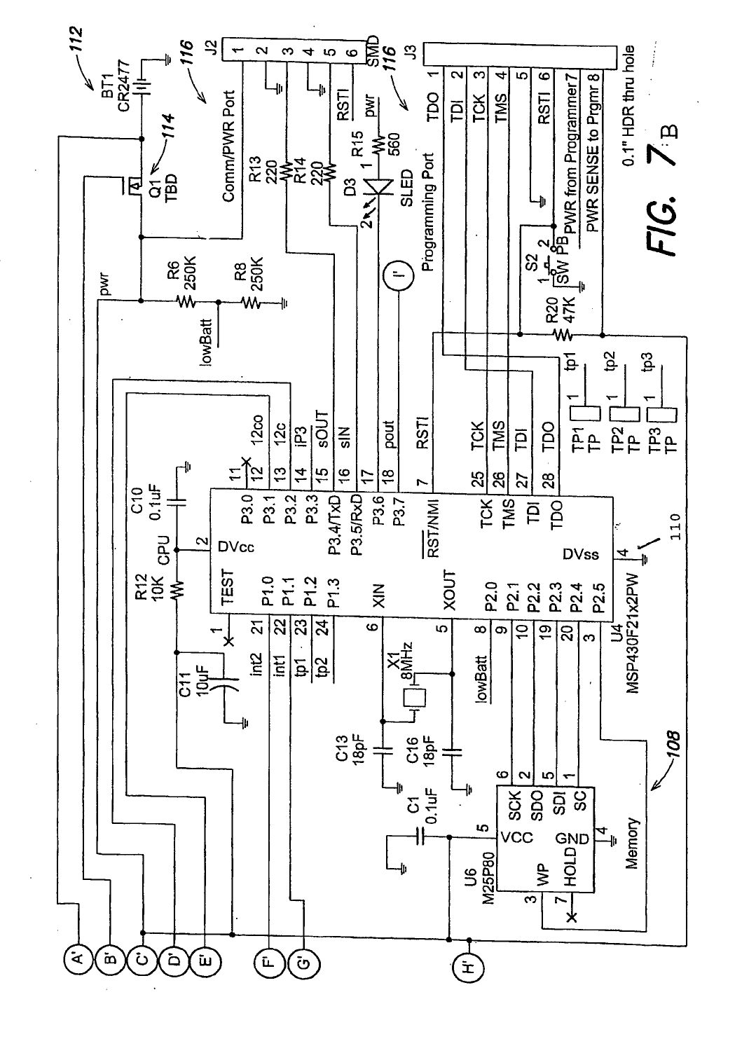

As shown in FIG. 1, the apparatus 100 also may include a processor 110 to receive the output signal(s) 106 generated by the sensing element(s) 104, a memory 108 (e.g., to store data relating to the output signal(s) 106), a communication interface 116 (e.g., to communicate information to and/or from the apparatus 100) and a power source 112 (e.g., to provide power to one or more components of the apparatus 100). The apparatus also may include switching circuitry 114, electrically coupled to the power source 112, to detect a proximity of the apparatus 100 to the person's head 50 and to electrically couple and/or decouple the power source 112 and at least the processor 110, based at least in part on the detected proximity of the apparatus to the person's head. Additional details relating to these various components are discussed below, for example in connection with FIGS. 6, 7A and 7B. Although FIG. 1 illustrates that all of these components may be disposed on or otherwise integrated with the flexible substrate 102, it should be appreciated that in other embodiments, one or more of the components other than the sensing element(s) 104 need not necessarily be disposed on or otherwise integrated with the flexible substrate 102.

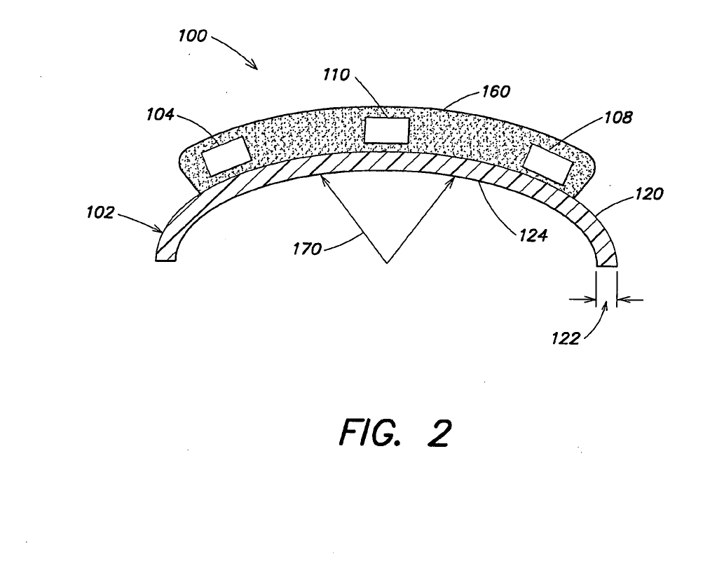

FIG. 2 illustrates a cross-section profile of the apparatus 100 of FIG. 1, according to one embodiment of the invention, wherein the apparatus is configured as a flexible tape 120. Due to the illustrative cross-sectional view, not all of the components shown in FIG. 1 are visible in FIG. 2—for purposes of illustration, only the sensing element(s) 104, the processor 110, and the memory 108 are illustrated as disposed on the flexible substrate 102, which is formed as a flexible tape 120. FIG. 2 also shows that the apparatus 100 may include an encapsulant 160 to encapsulate at least the sensing element(s) 104, and optionally other components of the apparatus as well. Regarding suitable encapsulants, generally one or more of the various materials discussed above that may employed for the flexible substrate 102 also may serve as the encapsulant 160.

In the embodiment of FIG. 2, the flexible tape 120 may be formed of any of the materials noted above in connection with the flexible substrate 102. In one aspect, the flexible tape 120 may be configured to have a thickness 122 on the order of approximately five millimeters or less. In another aspect, the thin flexible nature of the tape 120 provides for a significant bending radius 170 of the apparatus 100 to facilitate conformality to a variety of surface contours; for example, in one implementation, the apparatus 100 based on a flexible tape 120 may have a bending radius 170 in a range of approximately one centimeter to four centimeters. In yet another aspect, the flexible tape 120 may have an adhesive disposed on at least one surface 124 of the tape to render the tape “sticky” (so as to facilitate coupling of the flexible tape 120 to various surfaces). In yet another aspect, the flexible tape 120 may be configured, together with other components of the apparatus 100, to weigh on the order of one ounce or less.

Although FIG. 2 illustrates an example of the apparatus 100 in the form of a flexible tape 120, it should be appreciated that embodiments of the present invention are not limited in this respect. In general, the apparatus 100 may be implemented in a variety of form factors involving a flexible substrate, and having a variety of shapes and dimensions.

In some exemplary implementation of the embodiments shown in FIGS. 1 and 2, one or more of the various functional components of the sensing apparatus 100 (e.g., the sensing element(s) 104, the processor 110, the memory 108, the communication interface 116, etc.) may be a “commercial off-the-shelf” (COTS) component (e.g., a pre-packaged chip) that is disposed on or integrated with the flexible substrate 102. In particular, as discussed further below in connection with FIGS. 7A and 7B, in some implementations a particular COTS component may be single chip package that implements the combined functionality of one or more types of sensors, the processor, and/or the memory. Similarly, some COTS components may include amplifying circuitry, analog-to-digital conversion components, and/or other logic and circuit components. In other implementations, one or more functional components may be particularly-fabricated, and disposed on or integrated with the flexible substrate 102, for example, at a die level.

In view of the foregoing, it should be appreciated that not only does the conformality of the flexible substrate 102 of the apparatus 100 facilitate intimate proximity with the surface 52 of the head 50 (or other surface of interest in connection with which force and/or change of motion sensing is desired); additionally, in some examples, discrete functional elements in the form of COTS components or particularly-fabricated dies of sufficiently small size permit an appreciably small “footprint” of the apparatus 100 so as to facilitate conformality and sufficient mechanical coupling to a surface of interest. In at least some embodiments disclosed herein, a single sensing element (e.g., one pressure sensor, or one accelerometer), in some instances packaged together in a single COTS component with one or more of a processor, memory and other supporting circuitry, may be disposed on or otherwise integrated with the flexible substrate 102 to provide a conformal sensing apparatus having an appreciably small size (footprint).

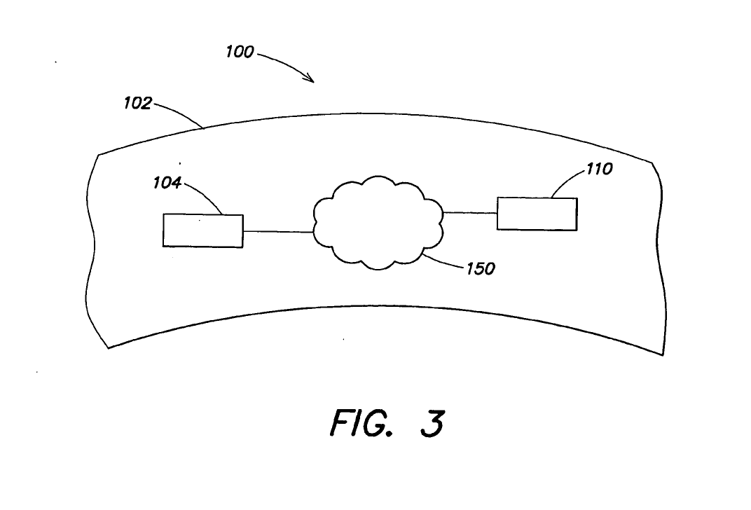

FIG. 3 illustrates a top view of the apparatus 100 of FIG. 1, according to another embodiment of the present invention, in which one or more functional electrical components of the apparatus (e.g., the sensing element(s) 104 and the processor 110) may be electrically connected by one or more flexible and/or stretchable interconnects 150. In one aspect of this embodiment, the use of flexible and/or stretchable interconnects 150 to electrically couple various components, together with the flexible substrate 102, may significantly enhance the conformal nature of the sensing apparatus 100.

Flexible and/or stretchable interconnects 150 may employ metals (e.g., copper, silver, gold, aluminum, alloys) or semiconductors (e.g., silicon, indium tin oxide, gallium arsenide) that are configured so as to be capable of undergoing a variety of flexions and strains (e.g., stretching, bending, tension, compression, flexing, twisting, torqueing), in one or more directions, without adversely impacting electrical connection to, or electrical conduction from, one or more functional components of the apparatus 100. Examples of such flexible and/or stretchable interconnects include, but are not limited to, wavy interconnects, bent interconnects, buckled interconnects, and serpentine patterns of conductors (the flexible and/or stretchable interconnects 150 are illustrated in FIG. 3 as a generalized cloud, as a wide variety of form factors are possible). In various configurations, the flexible and/or stretchable interconnects 150 may be stretchable, for example, up to at least 300%. Additional details of various examples of flexible and/or stretchable interconnects 150 are provided in U.S. publication No. 2010 0002402-A1, published Jan. 7, 2010, filed Mar. 5, 2009, and entitled “STRETCHABLE AND FOLDABLE ELECTRONIC DEVICES,” as well as other published references incorporated herein by reference (e.g., see SUMMARY section above).

In connection with various embodiments disclosed herein, one or more coupling mechanisms may be employed to mechanically couple the apparatus 100 shown in FIGS. 1-3 to a surface of interest (e.g., a body part of a person, such as a head) at which forces and/or changes in motion are to be sensed. In various examples, such a coupling mechanism preferably facilitates a sufficient mechanical coupling to the surface/object of interest to ensure accurate sensing of forces and/or changes in motion. As noted above, in some embodiments the apparatus 100 may be coupled to or otherwise integrated with various protective garments or accessories to comfortably and accurately sense forces and/or changes in motion associated with a person's head 50, as well as other body parts. In this context, as discussed above in connection with FIG. 2, an apparatus 100 including a flexible substrate 102 configured as a flexible tape 120 with an adhesive surface 124 may be integrated with various protective garments or accessories, wherein the adhesive surface 124 of the flexible tape 120 serves at least in part as the coupling mechanism for the apparatus.

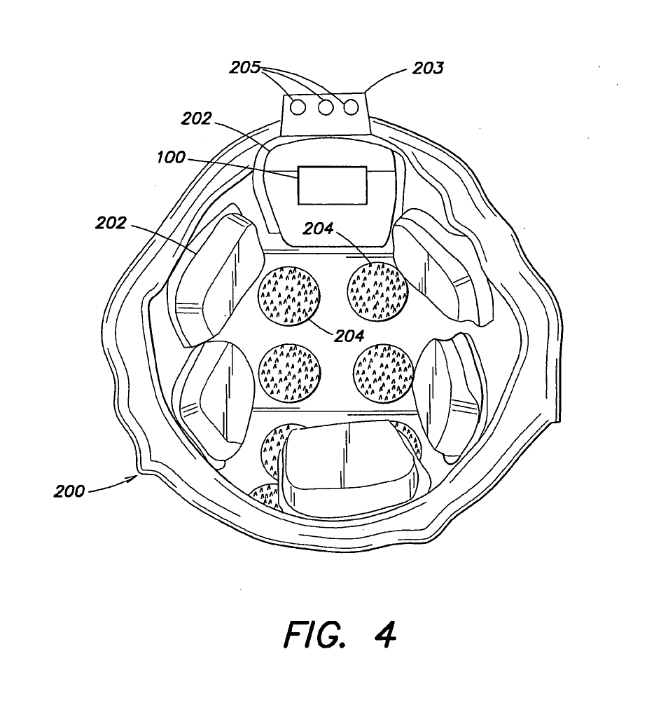

In other embodiments, a coupling mechanism for the apparatus 100 may take a variety of structural forms, and itself may include all or part of a protective garment or accessory, provided a sufficient mechanical coupling to a surface of interest is afforded by the coupling mechanism. To this end, FIG. 4 illustrates a helmet 200 including one or more suspension pads 202 with which the apparatus 100 of FIG. 1 may be integrated, wherein the system of the helmet and suspension pad(s) provide a suitable coupling mechanism between the apparatus 100 and the head 50. In particular exemplary implementations relating to military uses (e.g., to sense exposure to blasts and potential injury associated therewith), the helmet may be a combat helmet, such as an Army Modular Integrated Communication Helmet (MICH) or a Marine Corp Lightweight Helmet (LW).

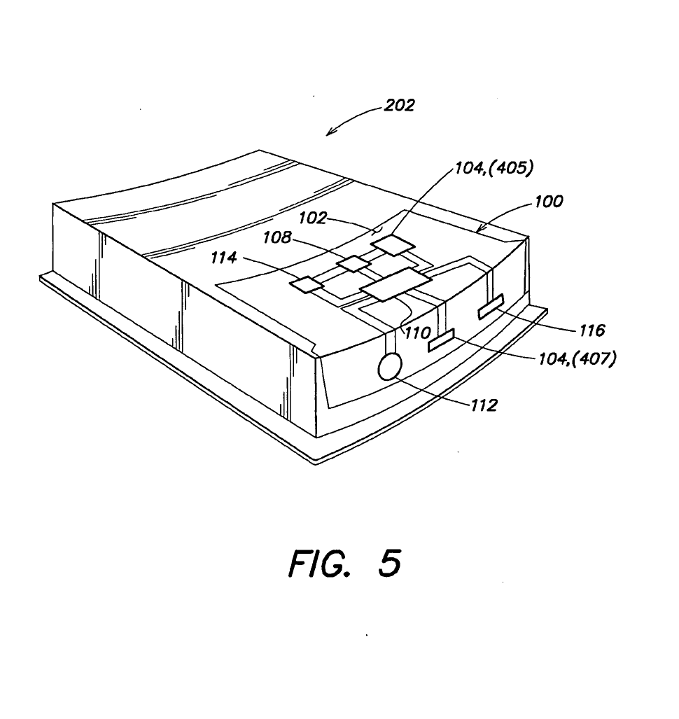

In FIG. 4, the helmet 200 may include one or more pad coupling mechanisms 204 (e.g., Velcro® brand hook-and-loop fasteners) to detachably couple the suspension pad(s) 202 to the helmet (e.g., so as to facilitate flexible placement of the suspension pad(s) on the helmet). As shown in FIG. 5, in one aspect of this embodiment, the apparatus 100 is integrated with a suspension pad 202 such that when the helmet 200 is worn by a person, the apparatus 100 is in comfortable and safe intimate proximity to the person's head 50 (e.g., to accurately sense a force and/or a change in motion at the surface 52 of the person's head). As illustrated in FIG. 5, in one exemplary implementation the apparatus 100 is affixed to or integrated with the suspension pad 202 so as to constitute at least a portion of a surface of the suspension pad 202, such that when the helmet is worn by a person, the apparatus 100 is in substantially direct contact with a surface of the person's head.

In another aspect of the helmet 200 shown in FIG. 4, the modular suspension pad/pad coupling mechanism combination allows the helmet to accommodate respective suspension pads in configurations according to both military guidelines and personal preferences. Such a flexible implementation reduces both restrictions on placement (front, back, side, or top of head) of one or more particular pads that include the apparatus 100, and also reduces the burden of retraining helmet wearers (e.g., soldiers) in connection with using the apparatus 100 (from the wearer's perspective, the presence of the apparatus 100 integrated with one or more suspension pads is essentially inconsequential to wearing the helmet and adjusting same for comfort and safety).

Additionally, after some period of time during which the apparatus 100 has been operating in the helmet and recording data relating to sensed force and/or change in motion (e.g., after 1 month, or some number of “blast events”), the suspension pad(s) 202 including the apparatus 100 can be easily detached from the pad coupling mechanism(s) 204, removed from the helmet 200, and opened to expose the communication interface 116 of the apparatus so as to access stored data. Alternatively, data stored in the apparatus 100 may be transmitted wirelessly to an external device (e.g., via a communication interface configured for wireless communication) for analysis/processing, as discussed further below in connection with FIGS. 6, 7A and 7B.

Referring again to FIG. 4, in one embodiment an output device 203 may be communicatively coupled to the apparatus 100 integrated in a suspension pad 202 of the helmet 200 to provide one or more perceivable indicators or cues (e.g., audible cues, visual cues) representing impact or trauma, based on forces and/or changes in motion sensed by the apparatus 100. For example, in one implementation, the output device may include one or more light sources 205 that provide different visual cues based at least in part on different degrees of sensed impact and/or potential trauma. Additional details of such output devices are discussed below in connection with FIG. 6.

FIG. 6 is a functional block diagram of the sensing apparatus 100 of FIG. 1, according to one embodiment of the present invention. FIG. 6 shows various functional components indicated in FIG. 1 (e.g., the sensing element(s) 104, the processor 110, the memory 108, the communication interface 116, the power source 112 and the switching circuitry 114), and indicates in greater detail that the sensing element(s) 104 may include an accelerometer 405 and/or a pressure sensor 407. As discussed immediately above, FIG. 6 also shows that a system based on the sensing apparatus 100 may include one or more output devices 203 to provide one or more perceivable indicators or cues (e.g., audible cues, visual cues) representing impact or trauma, based on forces and/or changes in motion sensed by the apparatus 100. In different implementations, as indicated by the dotted lines in FIG. 6, the output device(s) 203 may be coupled to the power source 112 (or receive power from a different source), and may be communicatively coupled to the processor 110 (e.g., either directly and/or via the communication interface 116).

FIGS. 7A and 7B illustrate a circuit diagram of the sensing apparatus of FIG. 1 corresponding to the block diagram of FIG. 6, according to one embodiment of the present invention. The various functional blocks indicated in FIG. 6 are mapped generally to corresponding circuit elements in FIGS. 7A and 7B. It should be appreciated that the circuit diagram shown in FIGS. 7A and 7B provides merely one implementation example of an apparatus and system based on the block diagram of FIG. 6, and that other implementations are possible according to other embodiments.

With reference to both FIG. 6 and FIG. 7A, regarding the sensing element(s) 104, in exemplary implementations the pressure sensor 407 may be an air pressure transducer, and in some instances an omni-directional air pressure sensor may be employed. In the case of a dynamic air pressure transducer, such a transducer may have a significant dynamic range (e.g., to sense pressure changes from whispers to blasts of explosive devices), represented by output signals 106 having a signal level in a range of from approximately 60 dB to 170 dB. Exemplary pressures represented by the output signal of a dynamic pressure transducer may be in a range of from approximately 4 pounds/square inch (PSI) to 100 PSI.

As shown in FIG. 7A, the pressure sensor 407 may include various circuitry associated with a pressure transducer, to condition signals generated by the transducer. For example, the associated circuitry may include an automatic gain control (AGC) amplifier, an analog-digital converter, and an adjustable resister to adjust a gain of the AGC amplifier. For example, the AGC range can be over 60 dB and the maximum gain can be set by a 22 MS resistance across the AGC amplifier (U5A). The resistor (R25) shown in FIG. 7A in series with the LED of the opto-coupler (D7, R23) dynamically adjusts the sensitivity of the AGC, by adjusting the current flowing through the opto-coupler based upon the output voltage of USB. Adjusting the current changes the brightness of the LED (D7), and in turn the resistance of variable resistor R23, which in parallel with R22, adjusts the gain of the AGC amplifier. The value of R22 (e.g., 22 MΩ) may be increased (to decrease the nominal gain of the AGC amplifier) if the feedback loop is unstable. The response time of the AGC feedback loop is sufficiently fast and the transducer allows for adjustment-free pressure sensing, even during an explosion (e.g., IED blast). With the wiper of bias resistor R21 set at 40K/60K (in a range of 0-100K) the sensing range of the pressure transducer is on the order of 4-100 PSI. The sensing range of the pressure transducer can be adjusted by changing the wiper position of R21. In various implementations, signal conversion between sound levels (dB) and PSI may be accomplished by the processor 110, or output signals from the pressure sensor may be transmitted to an external device (e.g., via the communication interface 116) for conversion of electrical signals to pressure levels (e.g., 100 PSI equals 170 dB, 50 PSI equals 132 dB, 4 PSI equals 89 dB).

Regarding the accelerometer 405, examples of accelerometers suitable for purposes of some implementations include any of the ADXL series of accelerometers (e.g., MEMS-based accelerometers) available from Analog Devices, Inc. (e.g., see http://www.analog.com/en/mems/low-g-accelerometers/products/index.html). Generally speaking, in some embodiments an accelerometer may be employed having a g-force rating that is significantly lower than the range of g-forces expected during typical use of the apparatus. For example, the selection of the g-force rating may in some cases be based on knowledge from the automotive industry (e.g., a car crash at around 25 MPH may generate a g-force significantly higher than 100 g, yet mass-produced automotive accelerometers used for airbag deployment typically are in the sub-50 g range). Additionally, in one aspect the accelerometer 405 may generate an analog output signal 106, and the analog-to-digital (A/D) conversion of the output signal 106 may take place elsewhere (e.g., in the processor 110, or in an external device); in another aspect, the accelerometer 405 may include integrated A/D conversion and provide a digital output signal 106.

As discussed above, the switching circuitry 114, portions of which are shown in both FIGS. 7A and 7B, is coupled to the power source 112 (e.g., see the battery BT1 in FIG. 7B) and electrically couples and/or decouples the power source and one or more other components of the apparatus 100. In one embodiment, the switching circuitry couples and decouples power to/from various components based on a detected proximity to a surface of interest for which sensed force and/or change in motion is desired (e.g., a body part). To this end, the switching circuitry may include one or more capacitive probes to detect a change in an electric field so as to detect the proximity of the apparatus to the body part. In particular, in some implementations, one or more capacitive probes detect an electrical conductivity of skin at the surface of the body part so as to detect the proximity of the apparatus to the body part.

With reference to the exemplary circuit diagram shown in FIGS. 7A and 7B, the switching circuitry 114 may be implemented by a “single key chip” given by the QTouch™ chip QT102 manufactured by Quantum Research Group (shown as U2 in FIG. 7A). This chip ultimately controls transistor Q1 (see FIG. 7B) to couple power provided by power source 112 (e.g., battery BT1) to the processor 110, the memory 108, and other components of the apparatus 100. A capacitive probe (see Sensor S1 in FIG. 7A) provides an input to the chip U2 based on detecting changes in electric field (e.g., associated with the conductivity of skin as the apparatus is placed in proximity to a skin surface). The change in electric field thus provides a “touch-on/touch-off” toggle mode for the switching circuitry 114. The circuitry 114 also may include other components (e.g., capacitors, resistors, and inductors) relating to timeout and timing override features.

As shown in FIG. 7B, the processor 110 and memory 108 may be implemented as respective COTS chips having any of a variety of appropriate features. In one exemplary implementation, the processor 110 can be a microcontroller unit (MCU) with the following specifications: Core Size 16-Bit; Program Memory Size 4KB (4K×8+256 B); Program. Memory Type FLASH; Connectivity SPI, UART/USART; Peripherals Brown-out Detect/Reset, POR, PWM, WDT; RAM Size 256×8; Speed 8 MHz; Number of I/O 22; Oscillator Type Internal; Data Converters A/D 8×10 b. Similarly, in one exemplary implementation, the memory 108 may have the following specifications: Memory Type FLASH; Memory Size 8 Mb (1 Mb×8); Speed 75 MHz; Interface SPI, 3-Wire Serial; Voltage-Supply 2.7 V˜3.6 V.

Regarding the communication interface 116 shown in FIG. 7B, in one example the communication interface may essentially be constituted by one or more ports providing connectivity to the processor 110. For example, the communication interface 116 may include a “programming port” for providing information to the processor 110, and a “Comm/PWR port” for connecting the power source 112 to one or more external devices, as well as providing two-wire transmit and receive signal capabilities to and from the processor 110 (see T×D pin 15 and R×D pin 16 of the processor 110).

More generally, it should be appreciated that the communication interface 116 may be any wired and/or wireless communication interface by which information may be exchanged between the apparatus 100 and an external or remote device, such as a remote computing device. Examples of wired communication interfaces may include, but are not limited to, USB ports, RS232 connectors, RJ45 connectors, and Ethernet connectors, and any appropriate circuitry associated therewith. Examples of wireless communication interfaces may include, but are not limited to, interfaces implementing Bluetooth® technology, Wi-Fi, Wi-Max, IEEE 802.11 technology, radio frequency (RF) communications, Infrared Data Association (IrDA) compatible protocols, Local Area Networks (LAN), Wide Area Networks (WAN), and Shared Wireless Access Protocol (SWAP).

Regarding the power source 112, in one exemplary implementation the power source may be a battery with the following specifications: Family Lithium; Series CR2477; Battery Cell Size Coin 24.5 mm; Voltage-Rated 3V; Capacity 660 mAh. In one example, the total power draw for the apparatus is configured to be approximately 67 μA per hour (assuming a 1 MHz sampling rate of the output signal 106 by the processor 110, with a 14-bit resolution). For an estimated average ‘ON’ time of about 20 hours per day, the battery life in this case would be about 41 days (in the event that the apparatus is stuck ‘ON’ for 24 hours per day, it will still last for 34 days with the foregoing exemplary ratings; also, if the sampling rate is decreased to <40 kHz, the battery size can be reduced even further).

Regarding the functionality of the processor 110 in exemplary embodiments, with reference to FIGS. 6, 7A and 7B, the processor 110 receives the output signal(s) 106 from the sensing element(s) 104 and, based on same, provides information relating to the sensed force and/or change in motion represented by the output signal(s). In particular embodiments relating to body parts, the information provided by the processor may relate to possible injury or trauma to a person resulting from the sensed force and/or changes in motion (e.g., information relating to possible head trauma).

More specifically, in one embodiment, the processor is configured to implement particular functionality via execution of processor-executable instructions stored in the memory 108, and/or internal memory of the processor 110. In one aspect, pursuant to executed instructions, the processor compares the sensed force and/or the change in motion represented by the output signal(s) 106 to at least one “trigger value” so as to provide the information relating to the possible injury/trauma. In various aspects, the trigger value(s) may represent one or more threshold values corresponding to particular forces or accelerations representing some type of force or impact event. For example, in one case, the trigger value(s) may represent a “significant blast event” in the context of an explosion, and the processor may be configured to identify the significant blast event by comparing the output signal(s) to the trigger value(s), and/or determine a blast exposure of the person subject to the explosion based at least in part on the trigger value(s) (i.e., the apparatus may serve as a “blast dosimeter”). In one particular example of a trigger value, the sensing element senses force, and the trigger value for force may be 40 pounds/square inch (PSI)). In another example, the sensing element senses acceleration, and the trigger value for acceleration may be 25 g.

In other aspects, one or more trigger values may be stored in the memory 108, and/or one or more trigger values may be received via the communication interface 116 (e.g., via the programming port shown in FIG. 7B) to facilitate downloading of a variety of trigger values based on different contexts/environments in which the apparatus 100 is to be employed. In another aspect, information provided by the processor 110 relating to the possible injury/trauma itself may be stored in the memory 108. In particular, one or more sampled and digitized output signals 106 themselves may be stored in the memory 108 for analysis/processing. To this end, the processor may be configured to sample the output signal(s) at a frequency up to approximately 1 MHz (e.g., so as to provide adequate sampling of acceleration over time to determine the severity of possible injury/trauma), and converts sampled analog signals to digital values via analog-to-digital conversion (e.g., in one example, the processor implements AID conversion having 14-bit resolution).

In one embodiment, the processor 110 controls the memory 108 so as to log and maintain in the memory the information relating to the possible injury/trauma if the sensed force and/or the change in motion represented by the output signal(s) 106 exceeds one or more trigger values. In one aspect, the processor logs the information into the memory for a predetermined time period (e.g., 5 seconds) after the sensed force and/or change in motion exceeds the trigger value(s). In another aspect, prior to exceeding one or more trigger values, the processor may continuously log information into a particular portion of memory, but merely over-write the logged data in the particular portion of memory to preserve memory resources until one or more trigger values are exceeded.

With respect to processing one or more output signal(s) 106, particularly in connection with sensing acceleration, in one embodiment the processor 110 may determine the information relating to possible injury/trauma based at least in part on an acceleration curve associated with the output signal(s) (e.g., integrating an output signal to determine an area under some portion of an acceleration curve). In one exemplary implementation, the processor may analyze an acceleration curve based at least in part on an automotive algorithm so as to determine the information relating to the possible injury/trauma. At least one example of such an automotive algorithm is provided in U.S. patent application publication No. 20100231401, published Sep. 16, 2010, and entitled “CONTROL DEVICE AND METHOD FOR TRIGGERING PASSENGER PROTECTION DEVICES,” which publication is incorporated by reference herein in its entirety.

With reference again to FIGS. 6 and 7B, although not shown explicitly in FIG. 7B, one or more output devices 203 may be coupled to the “Comm/PWR Port” of the circuit shown in FIG. 7B to receive control signals from the processor 110, and optionally power from the power source 112 (alternatively, the output device(s) 203 may include their own power sources). As noted above, in one embodiment, one or more acoustic speakers, and/or one or more light sources (e.g., LEDs), may be coupled as output devices 203 to the apparatus (e.g., and particularly to the processor 110) to provide one or more audible and/or visual cues representing impact or trauma. More specifically, based at least in part on one or more output signals generated by the sensing apparatus and provided as input to the processor 110, the processor in turn generates one or more control signals to appropriately control the output device(s) so as to provide indications based on sensed forces/changes in motion. In some implementations, the output device(s) 203 may be implemented as part of the apparatus 100 itself, or as a separate entity. Details regarding the integration of acoustic speakers and/or LEDs with a flexible substrate, which may be useful for some embodiments according to the present invention, are described in PCT application No. PCT/US2010/051196, filed Oct. 1, 2010, entitled “Protective Cases with Integrated Electronics,” and U.S. provisional application Ser. No. 61/247,933, filed Oct. 1, 2009, entitled “Protective Polymeric Skins That Detect and Respond to Wireless Signals,” both of which applications are incorporated by reference herein in their entirety.

In one exemplary implementation involving visual cues, multiple light emitting diodes (LEDs) having different colors are employed, wherein different colors of LEDs, when energized, respectively correspond to different degrees of the impact or potential trauma (e.g., red=high impact; orange: medium impact; blue: low impact). As shown and discussed above in connection with FIG. 4, such an output device may be integrated in some fashion with the helmet 200 (e.g., output device 203 with light sources 205, as shown in FIG. 4) to provide a local and simple yet instructive indication of possible injury (e.g., head trauma).

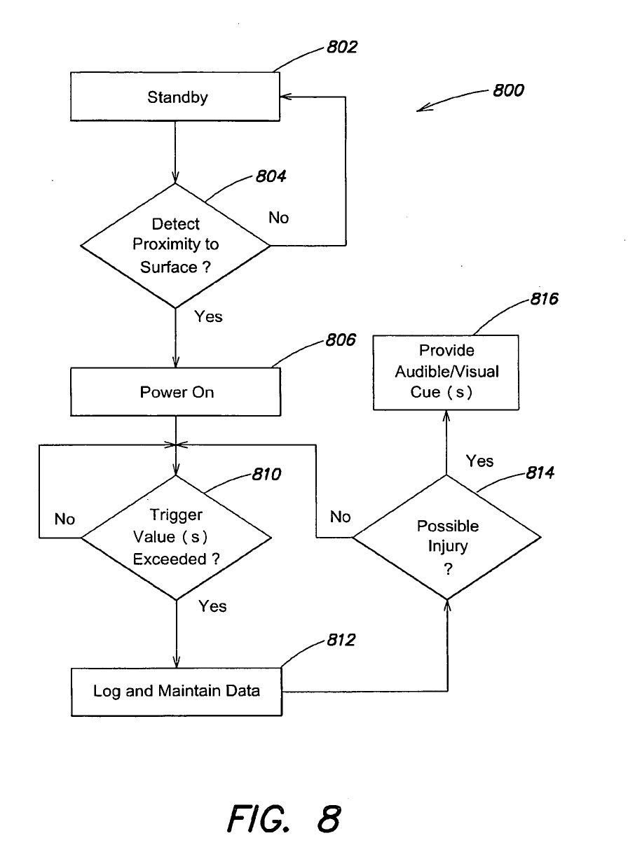

FIG. 8 is a flowchart illustrating a method 800 for conformal sensing of force and/or change in motion, according to one embodiment of the present invention. The method of FIG. 8 illustrates some of the salient respective functions performed by the apparatus 100 described above in various embodiments, when the apparatus is used in connection with a body part. It should be appreciated, however, that while the method outlined in FIG. 8 is directed to sensing force and/or change in motion in connection with a body part and providing information relating to possible injury based on same, the concepts disclosed herein regarding conformal sensing may be applied more generally to a variety of arbitrarily-shaped surfaces. Accordingly, methods similar to the one outlined in FIG. 8 may be applied, at least in part, for conformal sensing of force and/or change in motion proximate to surfaces of objects other than body parts.

In block 802 of the method 800 shown in FIG. 8A, the apparatus 100 is indicated in “standby” mode; i.e., the switching circuitry 114 has not detected proximity to a surface of interest, and hence power from the power source 112 is not yet applied to various components of the apparatus relating to sensing. In block 804, if proximity to a surface of interest is detected by the switching circuitry 114 (e.g., if a capacitive probe of the switching circuitry detects a change in electric field arising from proximity to skin at a surface of a body part), the switching circuitry 114 functions to couple power from the power source 112 to various components of the apparatus 100, as indicated in block 806 (“Power on”).

In block 810 of FIG. 8, one or both of a force (e.g., a pressure) and a change in motion (e.g., an acceleration) are sensed by the apparatus 100, and the sensed force and/or change in motion is compared to one or more trigger values for these parameters. As noted above, various trigger values may be selected to correspond to different types of anticipated events from which possible injury may result (e.g., trigger values associated with a blast or explosion; trigger values associated with a fall or other type of physical impact, etc.). If the sensed force and/or change of motion exceeds one or more trigger values, as indicated in block 812 the apparatus 100 begins to log and maintain data relating to the sensed force and/or change of motion.

In particular, as discussed above in connection with FIGS. 6, 7A and 7B, the processor 110 of the apparatus 100 may be configured to sample and digitize the output signals 106 generated by one or more sensing elements, and log the digitized sampled output signals in the memory 108. In one aspect, the processor may convert digitized sampled output signals to appropriate units representing force and/or change in motion (e.g., PSI and g-force) so as to compare these parameters to corresponding trigger values. In another aspect, the processor may be configured to log and maintain data in the memory for a predetermined period of time following one or more trigger values being exceeded (e.g., the processor may record data for 5 seconds following an event represented by the trigger value(s)). Otherwise, if one or more trigger values are not exceeded in block 810, the processor 110 may merely continue to monitor (e.g., sample and digitize) output signals representing sensed force and/or change in motion and store data accordingly in a prescribed portion of the memory 108, but continuously write-over stored data in the prescribed portion of memory until one or more trigger values are exceeded (so as to conserve memory resources).

In block 814 of FIG. 8, the processor 110 of the apparatus 100, and/or an external processing device coupled to the apparatus 100 (e.g., via the communication interface 116), may analyze the data logged pursuant to block 812 to provide information relating to possible injury of the body part based on the sensed force and/or change in motion. In one implementation, the mere fact that a sensed force and/or change in motion is identified by the processor as exceeding one or more trigger values itself establishes some degree of possible injury (e.g., a “significant blast event” may be identified as corresponding to one or more particular trigger values being exceeded). In other implementations, data relating to sensed force and/or change in motion may be analyzed to determine a “dose” or exposure to sensed force over some period of time, and/or changes in acceleration over time resulting from exposure to some force (e.g., an explosion or physical impact). As discussed above, in one embodiment, such analysis may be based at least in part on an acceleration curve (e.g., that is analyzed pursuant to one or more conventional automotive algorithms).

If possible injury is assessed in block 814 of FIG. 8, one or more audible and/or visual cues may be provided representing the possible injury, as indicated in block 816. As noted above, different cues may be associated with different degrees of possible injury; for example, in one embodiment, visual cues are provided as a plurality visual color light cues, wherein different color light cues respectively correspond to different degrees of impact or trauma (e.g., red=high impact; orange: medium impact; blue: low impact).

Conclusion

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments of the invention can be implemented in any of numerous ways. For example, some embodiments may be implemented using hardware, software or a combination thereof. When any aspect of an embodiment is implemented at least in part in software, the software code can be executed on any suitable processor or collection of processors, whether provided in a single device or computer or distributed among multiple devices/computers.

In this respect, various aspects of the invention, may be embodied at least in part as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium or non-transitory medium) encoded with one or more programs that, when executed on one or more computers or other processors, perform methods that implement the various embodiments of the technology discussed above. The computer readable medium or media can be transportable, such that the program or programs stored thereon can be loaded onto one or more different computers or other processors to implement various aspects of the present technology as discussed above.

The terms “program” or “software” are used herein in a generic sense to refer to any type of computer code or set of computer-executable instructions that can be employed to program a computer or other processor to implement various aspects of the present technology as discussed above. Additionally, it should be appreciated that according to one aspect of this embodiment, one or more computer programs that when executed perform methods of the present technology need not reside on a single computer or processor, but may be distributed in a modular fashion amongst a number of different computers or processors to implement various aspects of the present technology.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, the technology described herein may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

1. An apparatus for sensing force and/or acceleration proximate to a person's head, the apparatus comprising:
a flexible substrate to substantially conform to a surface of the person's head so as to facilitate comfortable and safe intimate proximity of the apparatus to the person's head;
at least one sensing element disposed on the flexible substrate, the at least one sensing element including at least one of a pressure sensor and an accelerometer and generating at least one output signal;
a processor, disposed on the flexible substrate and communicatively coupled to the at least one sensing element, to receive and process the at least one output signal; and
a memory, disposed on the flexible substrate and communicatively coupled to the processor, to store data relating to the at least one output signal.

2-132. (canceled)
